(19) United States
(12) Plant Patent Application Publication
(10) Pub. No.: US 2003/0051278 P1
(43) Pub. Date: Mar. 13, 2003

(54) Avocado tree named 'Merensky 2'

(76) Inventor: Josef Stefan Kohne, Duiwelskloof (ZA)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/046,490

(22) Filed: Oct. 25, 2001

Related U.S. Application Data
(60) Provisional application No. 60/317,871, filed on Sep. 7, 2001.

(51) Int. Cl. A01H 5/00

(52) U.S. Cl. PLT/200

(57) ABSTRACT

A new and distinct variety of avocado tree having many characteristics similar to those of ‘Duke 7’ that is characterized by strong resistance to Phytophthora cinnamoni, and which bears more fruits than the ‘Duke 7’ variety when ‘Hass’ is grafted thereon.

### RELATED APPLICATION

This application claims priority from Provisional Application Serial No. 60/317,871, filed Sep. 7, 2001.

### BACKGROUND OF THE INVENTION

The present invention generally relates to avocado trees. More particularly, the present invention relates to a new and distinct variety of avocado tree, Persea americana, with significantly improved resistance to root rot.

A seedling of the tree was discovered in an avocado orchard heavily infested with root rot, caused by Phytophthora cinnamoni at Westfalia Estate, South Africa. Most trees in that orchard died of root rot, however, the tree of the present invention was one of the few survivors and looked very healthy.

In the Westfalia nursery, an asexual reproduction of the tree rootstock was made by taking a bud bearing stick from a rootstock shoot and grafting onto a nurse seedling in the nursery. The grafted wood was subsequently rooted following standard procedures for producing clonal avocado trees. The trees were grafted with the scion ‘Hass’ in the nursery and planted in field trials in which the tree always showed the same superior resistance to root rot as the original seedling. The clones or propagules of the tree have been found to be identical to the original seedling in all distinguishing characteristics.

A Simple Sequence Repeat (SSR) DNA fingerprinting technique was performed on avocado leaves of various varieties, including the ‘Merensky 2’ . Three samples from the same cultivar were used. Aside from leaves of the ‘Merensky 2’, three other rootstock selections from the Westfalia nursery,

‘Edranol’, ‘Ettinger’, ‘Fuerte’, and ‘Duke 7’ were used. Total genomic DNA was isolated from the leaf samples using 2% CTAB and chloroform: isoamylalcohol (24:1). The SSR primers were: A1E11, A4F08, A7G04, of which the A7G04 primer was found unuseful and the A1E11 primer most useful in clearly distinguishing the cultivars, except two of the rootstocks from the Westfalia nursery. No variation could be found between the different samples from the same cultivar. It was concluded that two of the cultivars from the Westfalia nursery were identical. However, there were differences found between the ‘Merensky 2’ and the other varieties, and very clear differences found between the ‘Merensky 2’ and the ‘Duke 7’ variety.

In California, the ‘Merensky 2’ avocado trees have been planted and grown at the “University of California South Coast Field Station”, in Irvine, Calif.

### SUMMARY OF THE INVENTION

The invention relates to a new and distinct variety of an avocado tree having many characteristics similar to that of ‘Duke 7’. The invention is characterized by superior resistance to root rot, such as that caused by Phytophthora cinnamoni, as compared to ‘Duke 7’. The fruit of the present invention has been found to be slightly larger than that of ‘Duke 7’, and also has a better taste of that of ‘Duke 7’.

An additional report characteristic of ‘Merensky 2’ is its affect on the bearing of the ‘Hass’ avocado. ‘Hass’ grafted onto ‘Merensky 2’ bears more fruits than ‘Hass’ grown on ‘Duke 7’.

### BRIEF DESCRIPTION OF THE DRAWINGS

The accompanying photographs show the tree and fruit of the present invention. In such photographs:

FIG. 1 shows a ‘Merensky 2’ variety in South Africa;

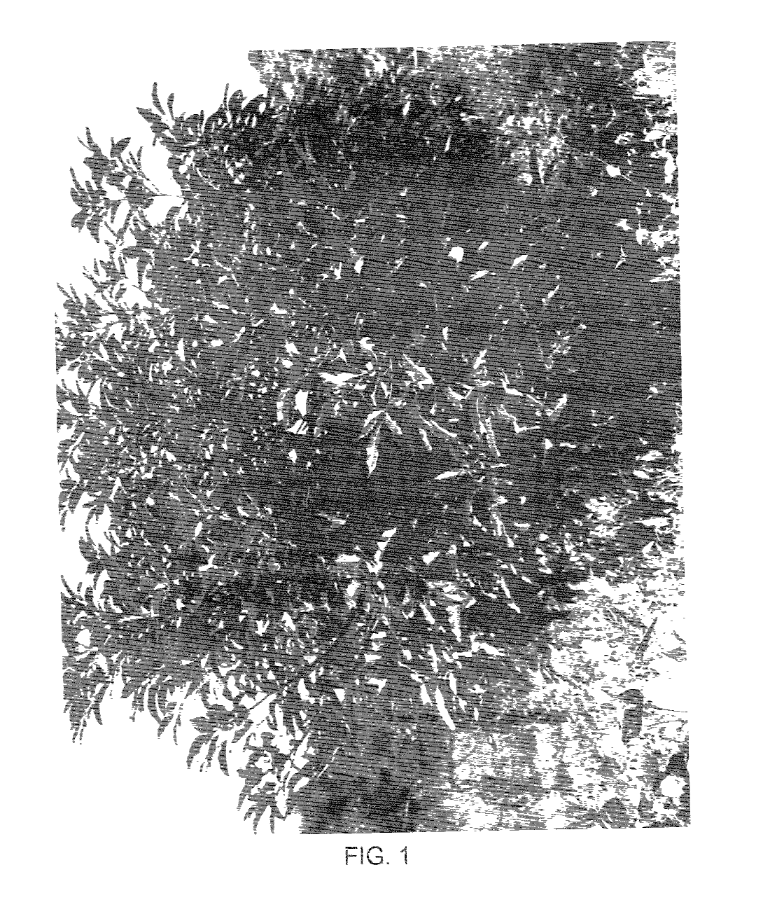

FIG. 2 shows a mature ‘Merensky 2’ in California;

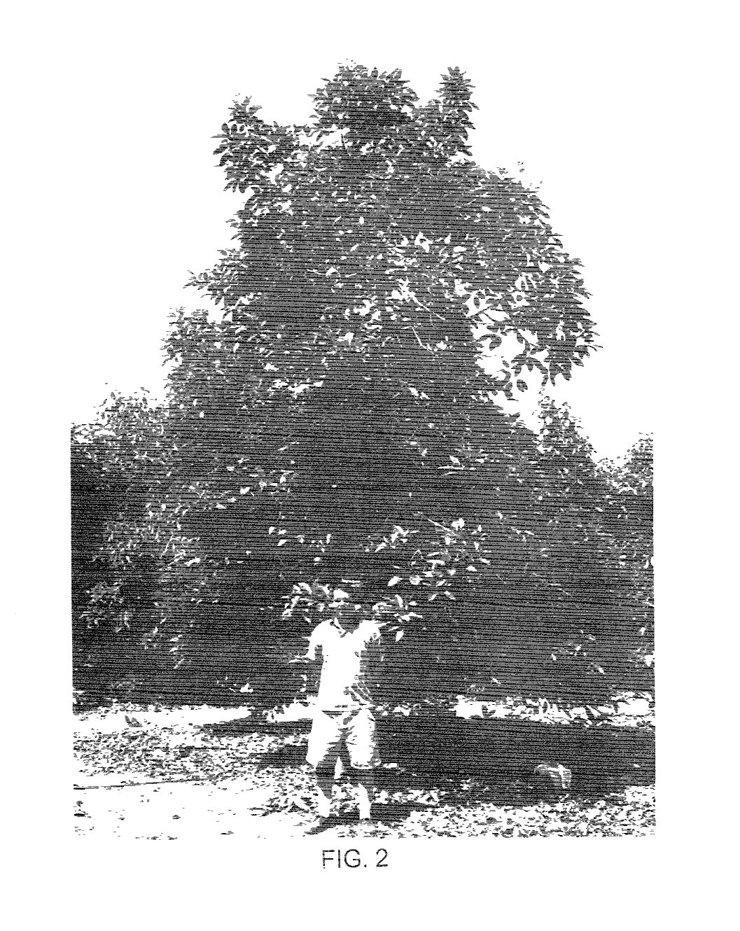

FIG. 3 shows flush from a ‘Merensky 2’;

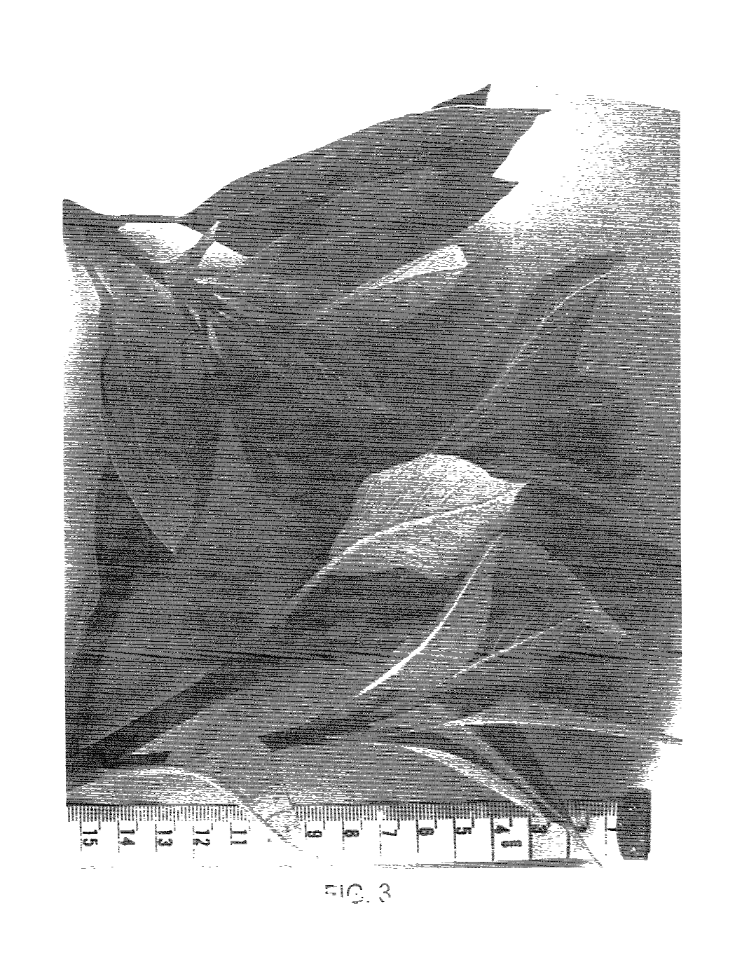

FIG. 4 shows flush from ‘Duke 7’;

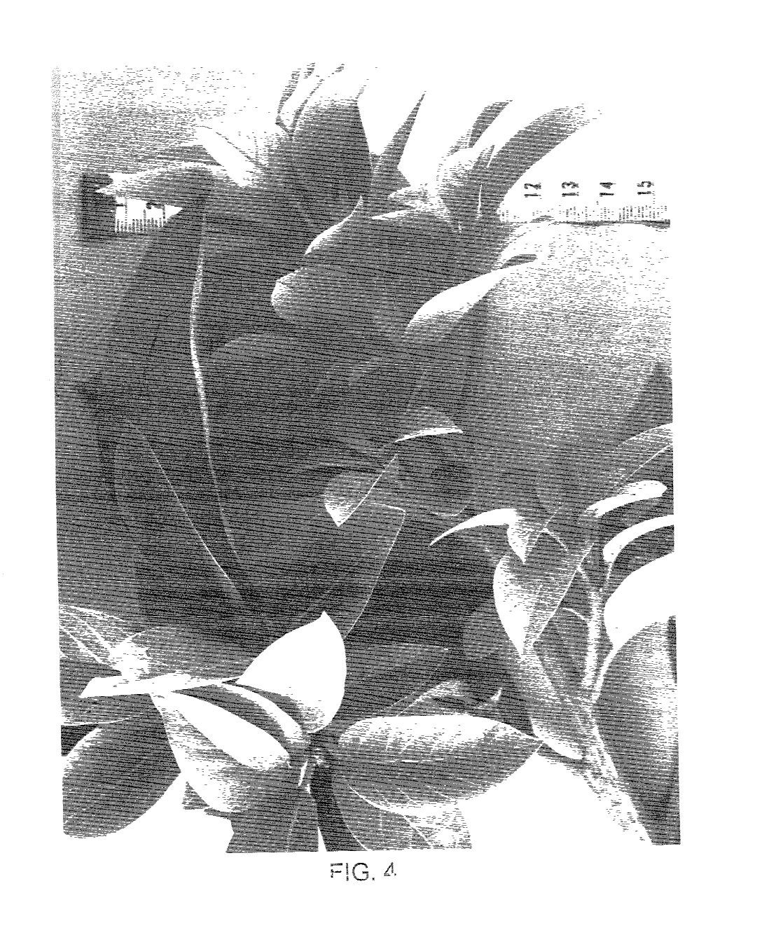

FIG. 5 shows a comparison of mature leaves of the ‘Duke 7’ (left) and ‘Merensky 2’ (right);

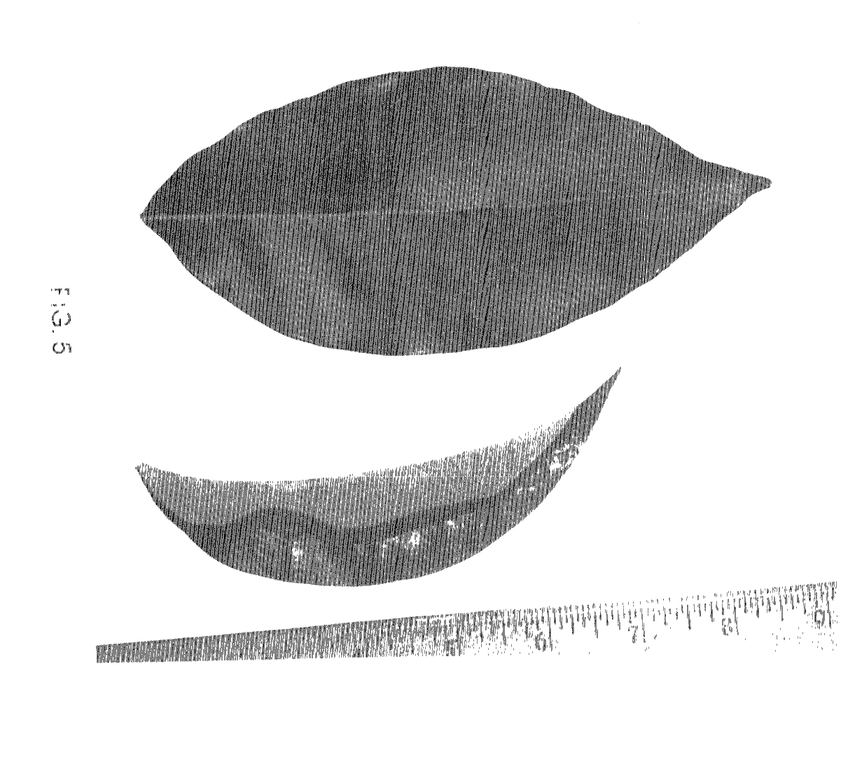

FIG. 6 shows a comparison of succulent stems of ‘Duke 7’ (left) and ‘Merensky 2’ (right);

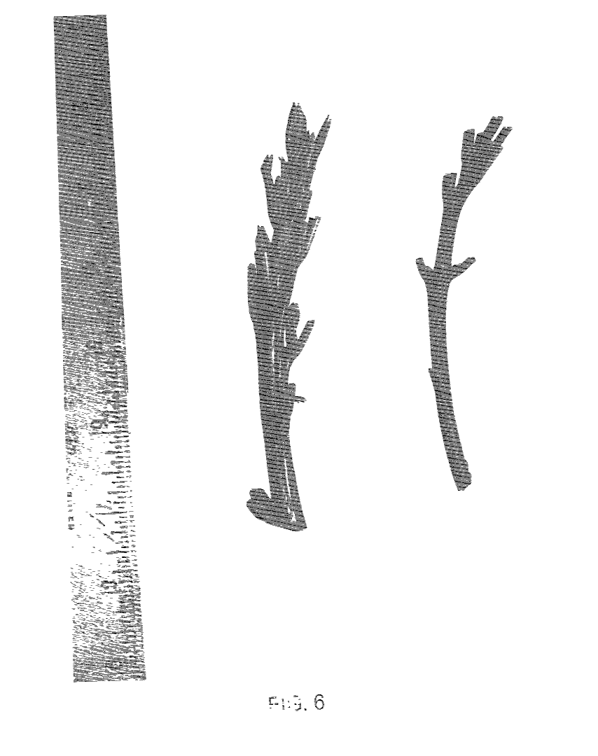

FIG. 7 shows the ‘Merensky 2’ in bloom;

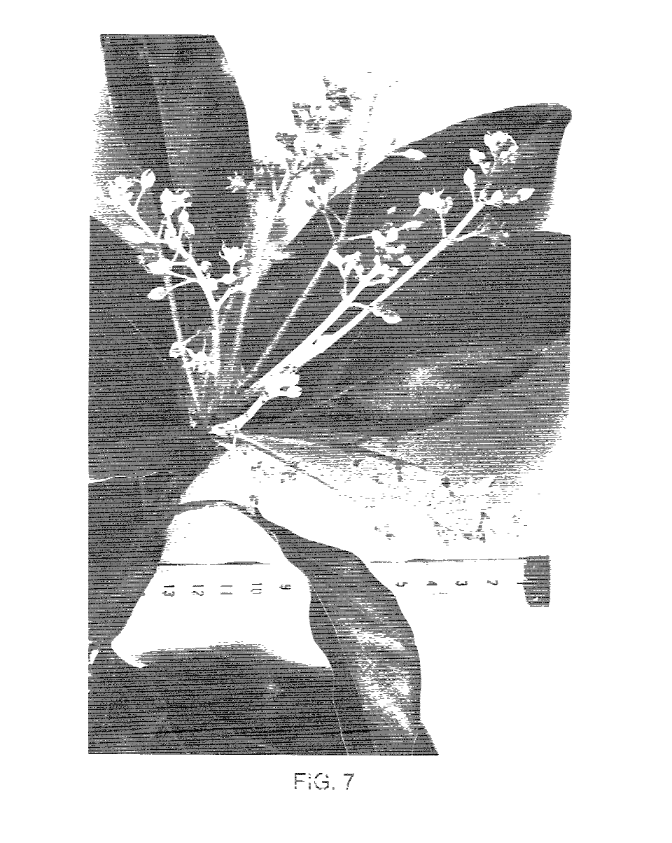

FIG. 8 shows the ‘Duke 7’ variety in bloom; and

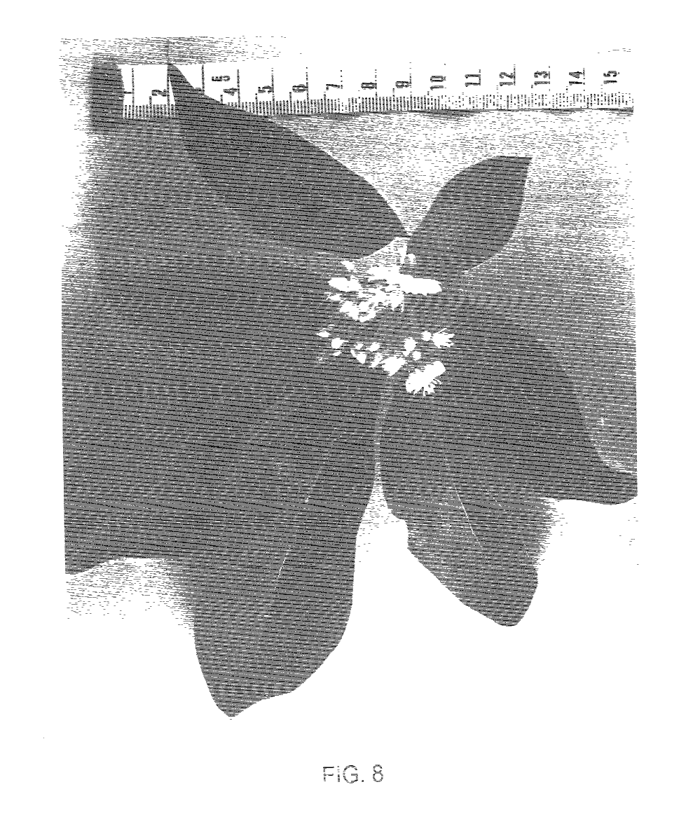

FIG. 9 shows an exteriorview of a mature fruit of the ‘Merensky 2’ variety.

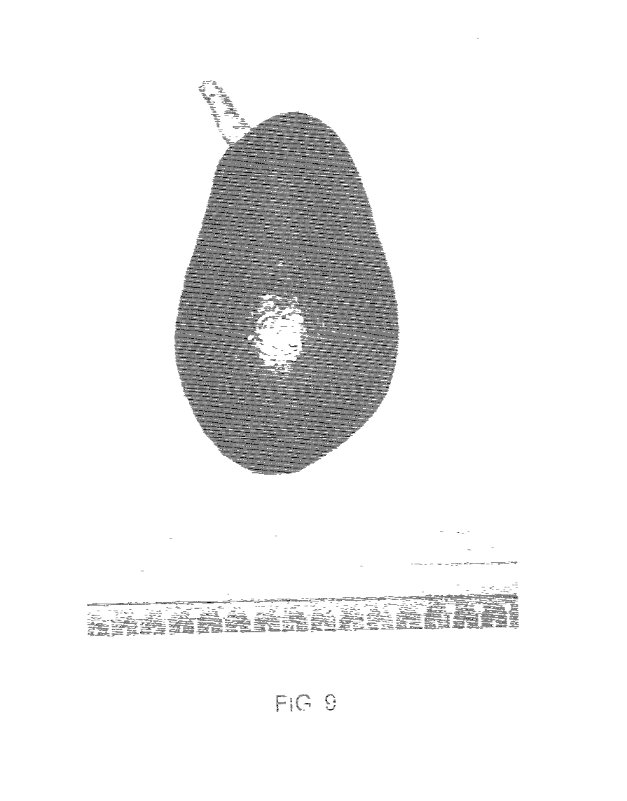

### BOTANICAL DESCRIPTION OF THE PLANT

In those instances where precise color assessment can be made, references are to the Royal Horticultural Society (RHS) color chart. In other instances, generally, color terms are used in accordance with an ordinary dictionary significance.

Cultivar Name: ‘Merensky 2’

Botanical Name: Persea americana

Form: Tree

Growth Habit: Medium vigor, medium size, with a spreading growth habit, whereas the ‘Duke 7’ is more upright.

Wood: One-year old branch: Normally green, smooth bark, having inconspicuous lenticels.

Main Stem:

Bark.—Greyish brown in color, having a corky texture.

Young shoot(Flush).—The intensity of anthocyanin coloration is medium, as compared to the absent or very weak coloration in ‘Duke 7’. The color of the flush is reddish (RHS 176A), whereas the ‘Duke 7’ is a light yellow-green (RHS 144B).

Lenticels.—The plant has medium conspicuousness of lenticels, whereas the ‘Duke 7’ variety is strong. The color of the lenticels in both the present variety and the ‘Duke 7’ is purplish.

Foliage:

Type.—Single leaf

Young leaf.—The present variety has an orange-brown (RHS 172A) color on its upper side, whereas the ‘Duke 7’ variety has a light orange-green (RHS 199A). The glossiness of the young leaf on flush is medium in the present variety.

Mature leaf.—The present variety has a length of approximately 17 centimeters and a width of approximately 7 centimeters, where the ‘Duke 7’ variety has a length of approximately 11.5 centimeters and a width of approximately 5.0 centimeters. Thus, the ratio of length to width in the present variety is 2.4, whereas in the ‘Duke 7’ variety it is 2.3. The shape of the leaves of both the present variety and ‘Duke 7’ variety is lanceolate to elliptic. The upper side of a mature leaf in the present variety and of the ‘Duke 7’ variety is of a medium gloss, having a dark green (RHS 147A) color. The color of the lower side of a mature leaf in the present variety is medium green (RHS 147B), whereas in the ‘Duke 7’ variety it is blue green (RHS N138B). Both the present variety and the ‘Duke 7’ variety have prominent veins on the lower side of the mature leaf. The mature leaf of the present variety is generally flat in cross section, whereas the ‘Duke 7’ is folded upwards. Whereas reflexing of the apex of a mature leaf is present in the ‘Duke 7’ variety, it is absent in the present variety. The colors of the petiole of the mature leaf is generally the same as the ‘Duke 7’ variety, that is yellow-green (RHS 145A). Similar to the ‘Duke 7’ variety, the mature leaf of the present variety has an anise aroma.

Fruit:

Quality.—Very good quality, whereas the ‘Duke 7’ variety can be watery.

Form.—The shape of the fruit of the present is obovoid, whereas the fruit of the ‘Duke 7’ variety is obovate.

Size.—The present variety of fruit has a length of approximately 11 centimeters, and a width of approximately 7 centimeters for a length to width ratio of 1.6. By comparison, the size of the mature fruit of the ‘Duke 7’ variety is 8.9 centimeters in by 5.5 centimeters in width, or the same ratio of length to width as the present variety.

Skin color.—When ripe, the fruit of the present invention has a very dark green (RHS 137A) skin color.

Skin texture.—The texture of the skin is similar to that of ‘Duke 7’ variety, and very smooth.

Longitudinal ridges.—Whereas longitudinal ridges are absent in the ‘Duke 7’ variety, one strong and long longitudinal ridge is present in the present variety.

Skin.—The skin of the present variety is very thin and membranous, and the adherence of the skin to the flesh of the fruit is very strong. In the ‘Duke 7’ variety, the skin is thin, but the adherence of the skin to the flesh of the fruit is weak.

Color.—The main color of the flesh of the fruit of the present variety is light yellow (RHS 154D) and, with the color of the flesh next to the skin being yellow-green (RHS 144A). The width of the more intensely colored area of the flesh next to the skin is approximately 3 millimeters. Fibers in the flesh are conspicuous.

Seed.—The seed of the present variety is approximately 4.8 centimeters in length and approximately 4.2 centimeters in width. By contrast, the seed of the ‘Duke 7’ variety is approximately 4.4 centimeters in length and approximately 3.4 centimeters in width. The seed shape (in longitudinal section ) of both the present variety and the ‘Duke 7’ variety are ovate. The color of a fresh seed coat of the present variety is orange-brown (RHS 165A).

Bloom: Similar to the ‘Duke 7’ variety, the present variety begins to flower and bloom in August, in South Africa. The flowering time in California has not been carefully studied, but is believed to blossom in late February to early May.

Harvest: In South Africa, the present variety is harvested in March, whereas the ‘Duke 7’ is harvested in February. Although not carefully studied in California, it is believed that the fruit will be harvested sometime in November to as late as May.

Disease: Whereas the ‘Duke 7’ variety has medium resistance to Phytophthora cinnamoni, the present variety is significantly more resistant to Phytophthora cinnamoni than the ‘Duke 7’ variety.

The tree and its fruit herein described may vary in ‘Merensky 2’, as herein described and illustrated.

What is claimed is:

1 An avocado tree as shown and described, characterized by superior resistance to root rot, and bearing more fruit when ‘Hass’ is grafted thereon than ‘Hass’ grown on ‘Duke 7’BROKAW -40124 PLANT PATENT
